(19) United States
(12) Patent Application Publication
(10) Pub. No.: US 2022/0011042 A1
(43) Pub. Date: Jan. 13, 2022

(54) CONTROLLED INVENTORY REFRIGERATED DISPENSING SYSTEM

(71) Applicant: Minibar North America, Inc., Rockville, MD (US)

(72) Inventors: Mark T. Cosgrove, Edgewater, MD (US); Paul Knox, Llandeilo (GB); Bruno Riegl, Seattle, WA (US); Alan K. Stratton, Manchester, NH (US); George Bonnoitt, Manchester, NH (US); Timothy Buckley, Columbia, MD (US)

(21) Appl. No.: 17/484,173

(22) Filed: Sep. 24, 2021

Related U.S. Application Data
(63) Continuation of application No. 16/118,940, filed on Aug. 31, 2018, now abandoned.
Application No. 16/118,940 is a continuation-in-part of application No. 15/629,890, filed on Jun. 22, 2017.
Application No. 15/629,890 is a continuation-in-part of application No. 14/259,621, filed on Apr. 23, 2014, now Pat. No. 9,734,303.
(60) Provisional application No. 62/552,673, filed on Aug. 31, 2017.
(60) Provisional application No. 61/815,045, filed on Apr. 23, 2013.

(51) Int. Cl. F25D 25/02; G06Q 10/08; G16H 20/13

(52) U.S. Cl. CPC F25D 25/02; A61J 1/165; G16H 20/13; G06Q 10/087

(57) ABSTRACT

A refrigerated dispensing system is provided including an outer refrigerator cabinet and an interior, removable chassis that mounts a plurality of separately removable product storage and dispensing cartridges and a separately removable open-front product storage box. While the storage-box and individual cartridges are removably positionable within slots on one or more shelves inside of the chassis, the entire chassis (with storage box and cartridges positioned thereon) may be removed from the outer refrigerator cabinet. The open-front storage box is provided RFID product tracking for monitoring inventory of products stored within the storage box.

### CROSS REFERENCE TO RELATED APPLICATION

This application is a continuation of U.S. patent application Ser. No. 16/118,940 titled “Controlled Inventory Refrigerated Dispensing System,” which application claims the benefit of U.S. Provisional Application No. 62/552,673 titled “Controlled Inventory Refrigerated Dispensing System,” filed Aug. 31, 2017 by the inventors herein. U.S. application Ser. No. 16/118,940 is also a continuation-in-part of copending and co-owned U.S. patent application Ser. No. 15/629,890 titled “Controlled Inventory Refrigerated Dispensing System,” filed Jun. 22, 2017, which application is a continuation-in-part of U.S. patent application Ser. No. 14/259,621 filed Apr. 23, 2014 and now U.S. Pat. No. 9,734,303, which application claims the benefit of U.S. Provisional Application No. 61/815,045 filed Apr. 23, 2013, all of which are incorporated herein by reference in their entireties.

### FIELD OF THE INVENTION

This invention relates generally to refrigerated product storage and dispensing, and more particularly to a refrigeration system for storing products (such as pharmaceutical products) and dispensing such products in a controlled manner that allows automatic tracking of inventory dispensed by and maintained within the refrigerated dispensing unit.

### BACKGROUND

Pharmaceutical products are distributed in many different ways. In many instances, the pharmaceutical product must be kept at a specific temperature in order to ensure its active ingredients retain their chemical properties. For example, some vaccines and antibiotics must be kept refrigerated to ensure that they maintain their pharmaceutical capabilities. In dispensaries, whether pharmacies or doctor's offices, these drug products are typically kept in traditional refrigerators. There is a minimum level of security and maintaining an inventory of the products can be a tedious and labor intensive endeavor.

Existing refrigerators and other such containers do not provide a doctor or pharmacist with an easy and secure method for dispensing medicines that require refrigeration. Thus, there is a need for a refrigerated container that allows physicians, pharmacists, and other individuals responsible for dispensing medicine to easily dispense the products and maintain an accurate inventory of the products.

Moreover, many systems for dispensing pharmaceutical products are maintained in a secure assembly, such as a locked cabinet, to prevent unauthorized access. However, in the event that such system is refrigerated, and must maintain its contents in a refrigerated condition, upon a loss of power it may become difficult if not impossible to remove product to alternative, refrigerated storage. Likewise, for systems that store many different pharmaceutical products, it may be a significantly time-intensive task to remove each pharmaceutical product from the dispensing system so as to place it in an alternative, refrigerated storage location. Thus, there is a need for a refrigerated container for pharmaceutical or other valuable products that allows quick, alternative access to locked contents during a loss of power, and that allows easy removal of such contents in bulk during such conditions.

Further, varied medical facilities have varying pharmaceutical products stored on hand from time to time, and thus have pharmaceutical product storage needs that may vary over time. Different pharmaceutical products may be provided in packages of varying sizes, such that one dispensing system for such products may not be suitable for all pharmaceutical products stored in any given facility. Thus, there is a need for a pharmaceutical or other valuable product storage and dispensing system that is adaptable so as to be able to securely store and dispense varied products over time.

### SUMMARY OF THE INVENTION

The present invention addresses one or more of the problems described above and other issues associated with traditional storage of refrigerated products that require a controlled inventory. A refrigerated dispensing system is provided including an outer refrigerator cabinet and an interior, removable chassis that mounts a plurality of separately removable product storage and dispensing cartridges, and a separately removable open-front storage box. While individual cartridges and the open-front storage box are removably positionable on one or more shelves inside of the chassis, the entire chassis (with cartridges and/or storage box positioned thereon) may be removed from the outer refrigerator cabinet, as may be desirable in the event of a loss of power to the outer refrigerator cabinet. The open-front storage box is provided RFID product tracking for monitoring inventory of products stored within the storage box.

With regard to a particular embodiment, a refrigerated storage and dispensing unit is provided, comprising: a refrigerator cabinet; a chassis positioned on an interior of the refrigerator cabinet having walls defining an open cavity on an interior of the chassis; at least one shelf affixed to the walls of the chassis within the open cavity; and an open-front storage box having a back wall panel, two side wall panels, a floor wall panel, and a top wall panel and defining an open interior space accessible through a front of the storage box, the storage box having at least one RFID antenna and at least one RFID reader, wherein the RFID antenna and the RFID reader are configured to read RFID tags on products positioned within the open interior space; wherein the open-front storage box further comprises a modular unit configured for insertion into and removal from the chassis.

### BRIEF DESCRIPTION OF THE DRAWINGS

The present invention and various aspects, features, and advantages provided by it are described in detail below with reference to exemplary and non-limiting embodiments and with reference to the drawings, which constitute part of this specification and provide graphic depictions of certain exemplary embodiments. The following is a brief description of the drawings:

FIG. 1 is a front perspective view of a refrigerated storage and dispensing unit in accordance with certain aspects of an embodiment of the invention.

FIG. 2 is a block diagram of the control and chilled compartment. FIG. 3A is a front perspective view of the refrigerated storage and dispensing unit of FIG. 1 with its front door open to provide access to the interior of the unit.

FIG. 3B is a front perspective view of the refrigerated storage and dispensing unit of FIG. 3A with an open-front storage box positioned on a top shelf and dispensing cartridges positioned on a bottom shelf.

FIG. 4 is a front perspective view of an interior chassis for use with the refrigerated storage and dispensing unit of FIG. 1.

FIG. 5 is an exploded view of the interior chassis of FIG. 4.

FIG. 6 is a close-up view of a chassis release system for use with the interior chassis of FIG. 4.

FIG. 7 is a front view of the chassis of FIG. 4.

FIG. 8 is a perspective view of a combined storage shelf and storage drawer for use with the chassis of FIG. 4.

FIG. 9 is a close-up view of the combined storage shelf and storage drawer of FIG. 8.

FIG. 10 is a front view of the combined storage shelf and storage drawer of FIG. 8.

FIG. 11 is a perspective view of the combined storage shelf and storage drawer of FIG. 4 with the printed circuit board and bottom plate removed for clarity.

FIG. 12 is a perspective view of the combined storage shelf and storage drawer of FIG. 11 with only the printed circuit board removed for clarity.

FIG. 13 is a side perspective view of the combined storage shelf and storage drawer of FIG. 11.

FIG. 14 is a top perspective view of the combined storage shelf and storage drawer of FIG. 11.

FIG. 15 is a rear perspective view of the combined storage shelf and storage drawer of FIG. 11.

FIG. 16 is a close-up rear perspective view of the combined storage shelf and storage drawer of FIG. 11.

FIG. 17 is a front perspective view of a dispensing cartridge for use with the storage and dispensing unit of FIG. 1.

FIG. 18 is a rear perspective view of the dispensing cartridge of FIG. 17.

FIG. 19 is a cross sectional view of an RFID open-front box for use with the storage and dispensing unit of FIG. 1.

FIG. 20 is a rear view of the RFID open-front box of FIG. 19.

FIG. 21 is a front perspective view of an RFID open-front box for use with the storage and dispensing unit of FIG. 1.

FIG. 22 is a rear perspective view of the RFID open-front box of FIG. 21.

FIG. 23 is a front perspective view of the RFID open-front box of FIG. 21 with shelves and door removed for clarity.

### DETAILED DESCRIPTION

The invention summarized above may be better understood by referring to the following description, which should be read in conjunction with the accompanying claims and drawings in which like reference numbers are used for like parts. The following description is of a particular embodiment of the invention, set out to enable one to practice an implementation of the invention, and is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

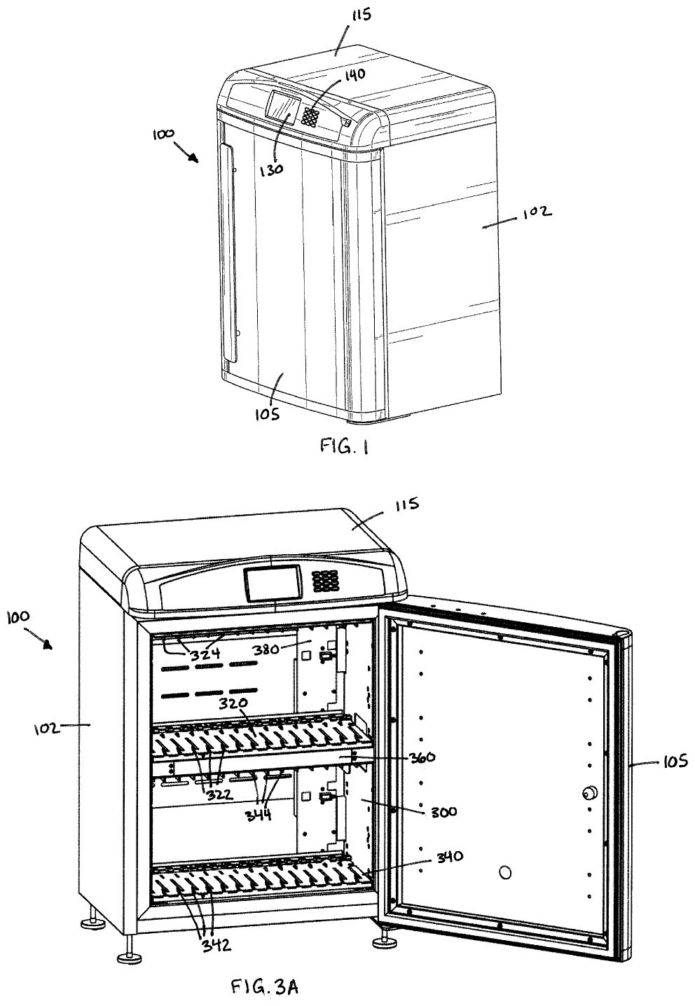

First, with reference to FIG. 1, an exemplary refrigerated storage and dispensing unit 100 according to certain aspects of an embodiment of the invention consists of a refrigerator cabinet 102 having a door 105 and a control compartment 115, which control compartment 115 may include a communication interface configured to communicate with a remote server. Refrigerator cabinet 102 may include a chiller (most preferably ammonia absorption, but in certain embodiments a compressor will be used).

A system utilizing such refrigerated storage and dispensing unit may be operated by a person removing a product from an interior of refrigerator cabinet 102, and control compartment 115 recording the removal of product as a transaction in its local memory on the control compartment 115. Periodically, the control compartment 115 may use the communication interface to send transaction data along with events (e.g., historical temperature data, power outages, events causing alarms, etc.) to a remote server. The remote server may then communicate with outside entities for inventory control, maintenance, alarms, billing, and any other essential tasks. A refill technician or specialist may communicate with the refrigerated storage and dispensing unit 100 using a display 130 and keypad 140 on the control compartment 115, or optionally via a web-enabled device, BLUETOOTH device, or other remote device to manage the functions and refilling of the refrigerated storage and dispensing unit 100.

Preferably, external door 105 is provided an electric or mechanical lock, and is used to control access to the products stored in the refrigerated storage and dispensing unit 100. If an electric lock is provided, such electric lock is preferably controlled by a computer processor in the control compartment 115 after a valid access code is entered or, as described more fully below, when specific events occur that require that the door be locked or unlocked.

Control compartment 115 provides a user interface that a pharmacist, doctor, or other user may engage to manage the refrigerated storage and dispensing unit 100. In accordance with certain aspects of an embodiment of the invention and as noted above, control compartment 115 preferably includes a display 130 and a user input device 140. Display 130 may consist of a LCD, and the user input device may be a key pad. Optionally, the user input device 140 and the display 130 may be combined into a touchscreen as will be recognized by a person of ordinary skill in the art. The control compartment 115 may also optionally include one or more of the following elements: an alarm sounder, a camera for a bar code reader, a USB communications interface, an external communications interface (Ethernet, PLC, POTS, Cellular, Satellite, WiFi, etc.), and an internal communications interface (I2C and GPIO) for communication among the internal components of the refrigerated storage and dispensing unit, all controlled by a computer processor.

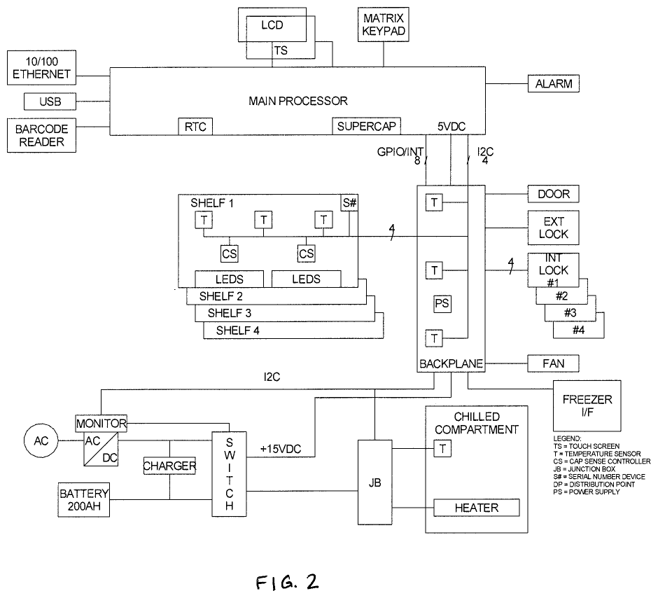

Control compartment 115 preferably includes the computer processor, memory, RTC, battery backup, and necessary interfaces to communicate with all hardware. The computer processor may use a Linux OS with an Android OS and application running on it, although alternative operating systems may be employed. FIG. 2 provides a schematic diagram of the electrical connections of an exemplary control compartment 115.

Preferably, all control and communications are managed by control compartment 115. Users communicate with the vending refrigerator 100 via the user interface, e.g., keypad 140 and LCD 130, touchscreen, or web or BLUETOOTH enabled device such as a smart phone, tablet, laptop, etc. Communications to a server and external users are accomplished via the external communications interface. The user interface may be managed through a mobile device application to control the screens, touchscreen and keypad. The same mobile application or a remote website can be used in a mobile device and, thus, the user does not have to be co-located with the refrigerated storage and dispensing unit 100 in order to affect such control. The mobile application or remote website enables any wireless communication device to interact with the refrigerated storage and dispensing unit 100 or the system that includes the refrigerated storage and dispensing unit 100.

An alarm sounder may be used to signify to local users that a critical event (e.g., no power, temperature out of range, etc.) has occurred and that a user is needed to attend to the refrigerated storage and dispensing unit 100. The user can mute the alarm, e.g., via LCD 130 and/or keypad 140. A USB interface preferably provides a maintenance port for diagnostics and emergency download of data. It is contemplated that the interface may be any port, USB or otherwise, that allows physical electronic access to the internal components of the refrigerated storage and dispensing unit 100, e.g., Ethernet port, HDMI, etc. Such interface may also be used to setup the refrigerated storage and dispensing unit 100 and for manufacturing assistance. A barcode reader, which consists of a camera, camera interface, and an application to manage the reader, e.g., a mobile device or wireless device, can be used for reading data from the products to be stored in the refrigerated storage and dispensing unit 100. Typical data may include the kind of product, expiration date and lot code.

The main power supply is preferably monitored to determine the type of current being supplied, e.g., AC or DC, the current voltage, and the current battery status. The interface is also used to control the switch between AC and a battery source for testing the battery and during loss of AC power. The switch is also used to shed the load of the chiller if the battery supply runs low. A backup power supply on the processor board consisting of a battery or supercapacitor is used in the event that the main power source is not present. In the event that the main power source is not present, software executable by control compartment 115 will load the volatile data into non-volatile memory (Flash, MRAM, FRAM, EEPROM, etc.) using this backup power supply.

Internal communications among the components of the refrigerated storage and dispensing unit 100 (discussed in detail below) may be managed via I2C of GPIO. The I2C is preferably used for the main communications for control and status. The GPIO is preferably used for real time interrupts and programming.

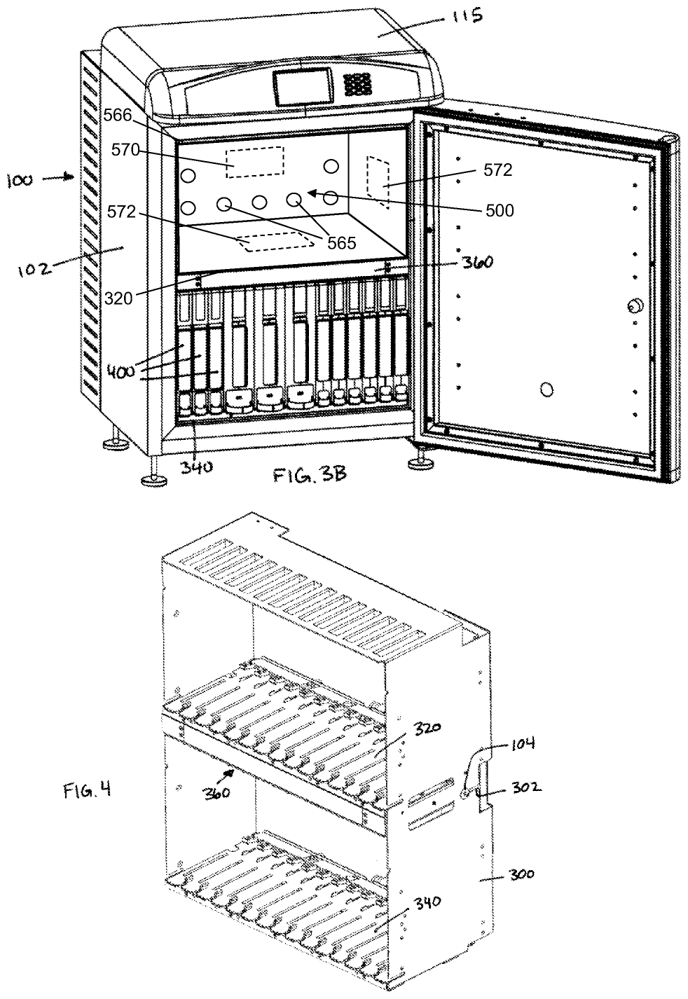

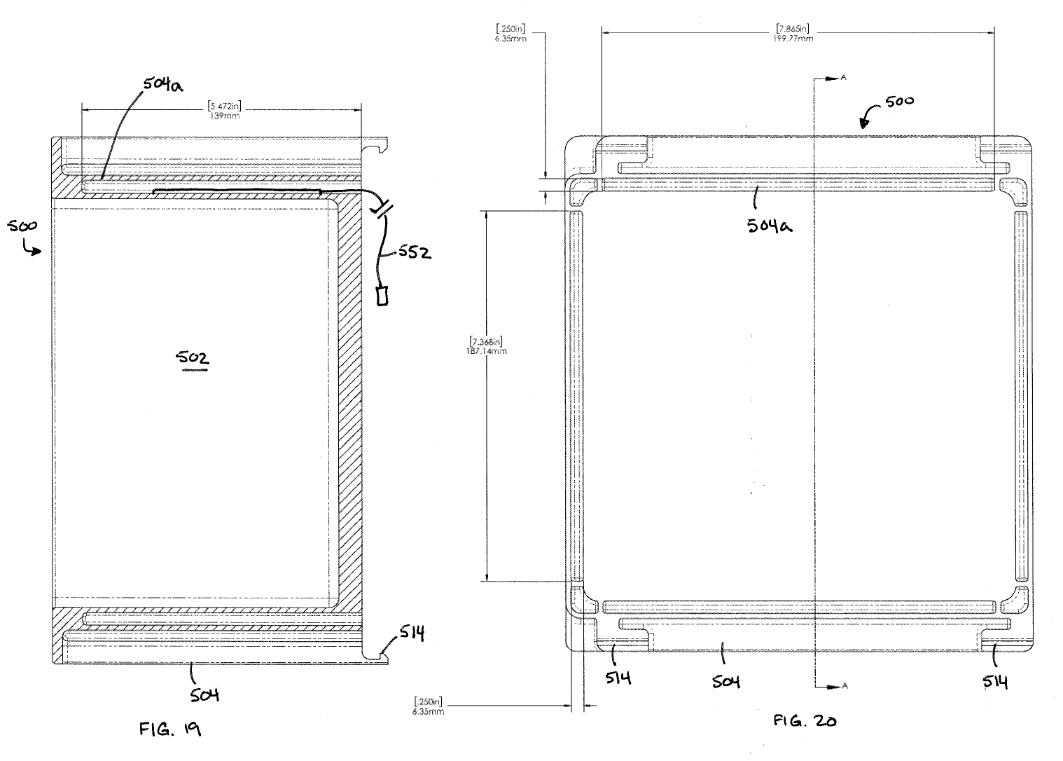

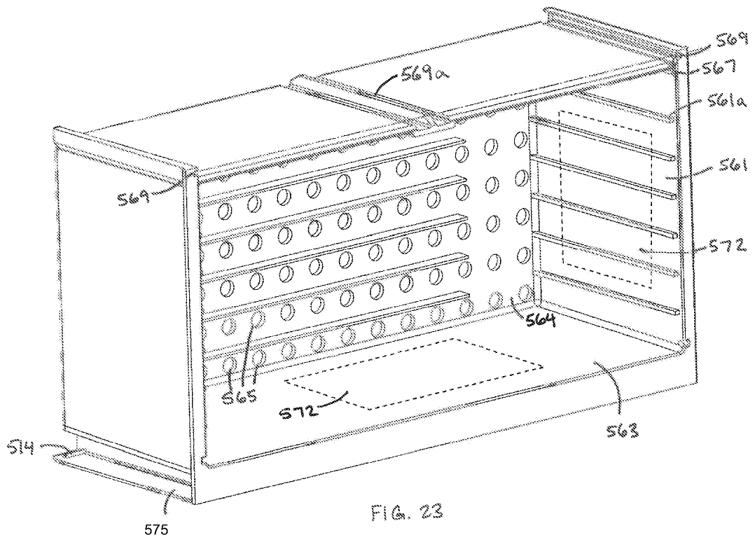

FIGS. 3a and 3b show door 105 of refrigerator cabinet 102 open to provide access to product stored within the refrigerated storage and dispensing unit 100. As shown in FIG. 3a (in which the open-front storage box and dispensing cartridges are not shown on shelves 320 and 340 for clarity) and in accordance with certain aspects of an embodiment of the invention, the refrigerated storage and dispensing unit may include an interior chassis 300, a top shelf 320, a bottom shelf 340, and an intermediate drawer assembly 360, all of which are discussed in greater detail below. Top shelf 320 and bottom shelf 340 define parallel slots configured to receive individual product dispensing cartridges 400 (FIG. 3b), with such product dispensing cartridges arranged parallel to one another along their respective shelf. More particularly, each such product dispensing cartridge 400 holds a plurality of product containers, such as vials of injectable pharmaceuticals, syringe packages, or other such packages as may be suitable to a particular facility or configuration. Moreover, each such product dispensing cartridge 400 includes a dispensing drawer at the base of each cartridge, with each such dispensing cartridge 400 being configured to dispense only a single product container at a time from its respective dispensing drawer. The product dispensing cartridges 400 are insertable into slots on shelf 320 and shelf 340. Such slots for top shelf 320 are defined by guide tabs 322 extending upward from shelf 320 and dividers 324 extending downward from an upper divider wall defined by the top wall of chassis 300, and slots for bottom shelf 340 are defined by guide tabs 342 extending upward from shelf 340 and dividers 344 extending downward from a lower divider wall defined by the bottom wall of intermediate drawer assembly 360. Further and as discussed in detail below, product dispensing cartridges 400 may be locked in place on shelf 320 and on shelf 340 with movable latches that engage the rear of each product dispensing cartridge 400. FIG. 3B shows an open-front storage box 500 (more specific details of which are shown in FIGS. 19-23 and the related discussion thereof below) positioned on top shelf 320, and a plurality of product dispensing cartridges 400 positioned on bottom shelf 340, with each of open-front storage box 500 and dispensing cartridges 400 being removable from its respective shelf 320/340 as discussed further within this specification.

Each of top shelf 320 and bottom shelf 340 preferably includes sensors that engage with product dispensing cartridges 400 (shown in FIG. 3b) to detect dispensing of product from such dispensers 400. In order to collect such data from top shelf 320 and bottom shelf 340, a backplane 380 is located at the back of chassis 300, and dispensing shelves 320 and 340 (and, as discussed below, RFID-enabled storage box 500 of FIGS. 21-23) electronically connect to backplane 380 to transfer data collected by sensors on dispensing shelves 320 and 340 to control compartment 115. Backplane 380 buffers the communication signals between the top shelf 320 and bottom shelf 340 and the processor of control compartment 115. Temperature sensors on backplane 380 may provide temperature data to the processor of control compartment 115. Controls for a fan and door locks are preferably located on backplane 380 and are in communication with the processor of control compartment 115. The door sensor logic is preferably stored on backplane 380 for the processor of control compartment 115 to access. Control logic on backplane 380 may determine which shelf, device, or freezer interface the processor platform communicates with. Power from the main power supply is distributed on backplane 380 for shelves 320 and 340 and logic. Power is preferably stepped down on backplane 380 for the processor in control compartment 115.

As mentioned above, top shelf 320 and bottom shelf 340 receive product dispensing cartridges 400, with such product dispensing cartridge sitting on top of a shelf 320 or 340, with contacts on the movable dispensing drawer of each product dispensing cartridge positioned and otherwise configured to activate the sensors on the shelf. In accordance with certain aspects of an embodiment of the invention, each of shelf 320 and 340 has several sensor pads under each product dispensing cartridge 400. More particularly, and by way of non-limiting example, each of shelf 320 and 340 may have at least three sensor pads facing upwards in a configuration that allows product and drawer sensing. The sensor pads are connected to a processor on the respective shelf 320 or 340, or directly to the processor of control compartment 115 through backplane 380. The sensors can be, by way of non-limiting example, capacitive, infrared, or mechanical. A sensing application running on the processor detects the presence or absence of product on that particular sensor, and may also detect the mechanical position of different parts of the product dispensing cartridge. This data is sent to the processor of control compartment 115 to determine if the product has been removed or if there is a malfunction. Data from a digital temperature sensor is also sent to the processor of control compartment 115 to be used in controlling a heater/cooler, for historical data for product aging, and for alarms.

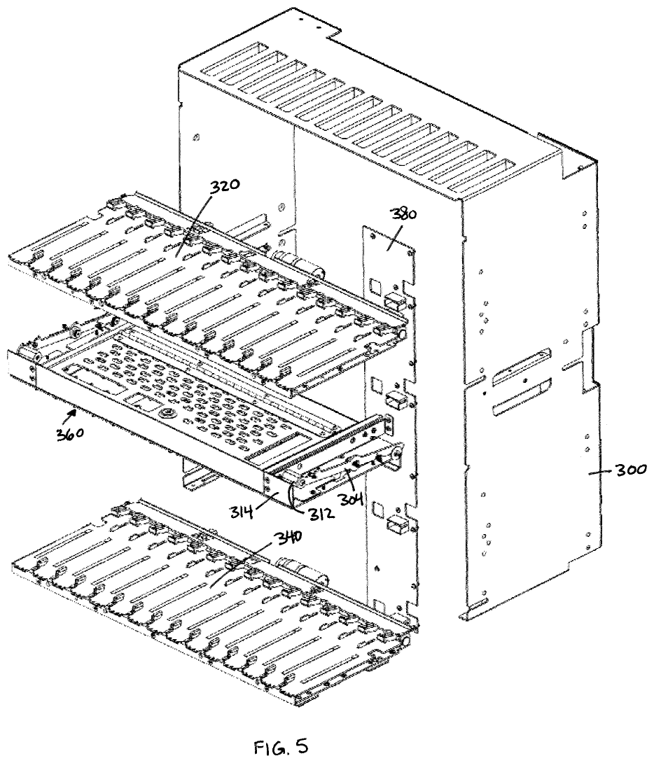

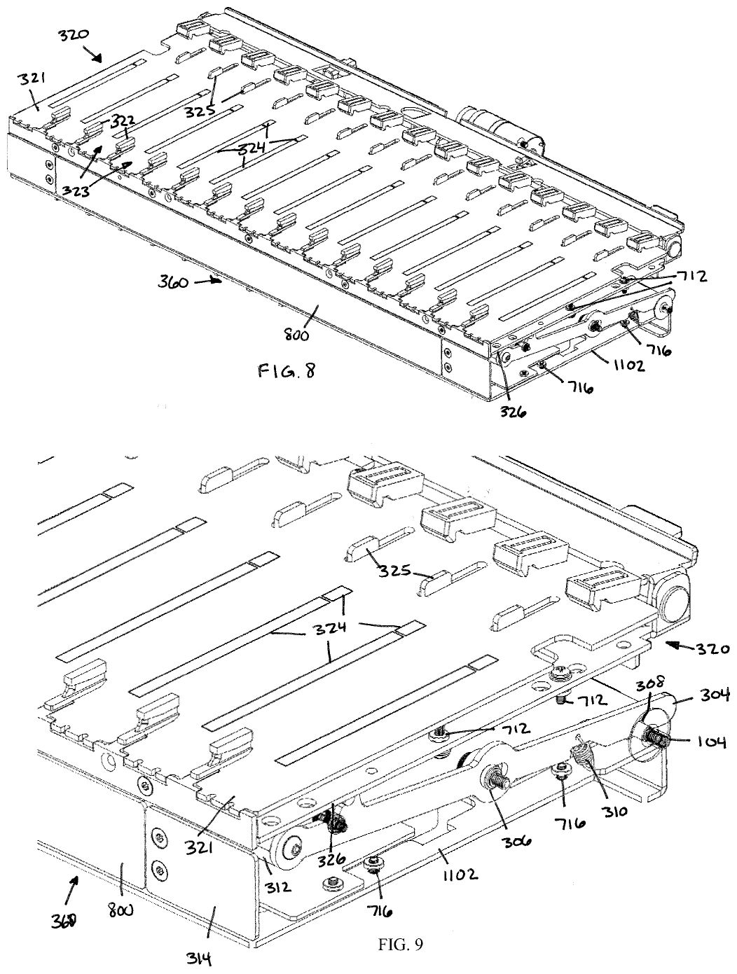

FIG. 4 provides a perspective view, and FIG. 5 an exploded perspective view, of chassis 300 removed from refrigerator cabinet 102 (refrigerator cabinet 102 not shown for clarity). Circumstances may arise that create a desirability or need to remove the entire contents of the refrigerated storage and dispensing system 100, such as in the event of a long-term power loss which would require moving all contents to an alternative refrigerated storage. To provide for such capability, the entire chassis 300 is removable from the interior of refrigerator cabinet 102. A refrigerator cabinet mounting nut 104 is fixedly mounted to at least one, and preferably both interior sidewalls of refrigerator cabinet 102. Chassis 300 has a horizontal mounting nut slot 302 in preferably both sidewalls of chassis 300, which mounting nut slot 302 slidably receives refrigerator cabinet mounting nut 104. As shown in FIG. 5 and FIG. 6 (in which chassis 300 is shown in phantom), mounting nut 104 serves as an anchor to releasably hold chassis 300 in refrigerator cabinet 102. A chassis release bar 304 is pivotably mounted to intermediate drawer assembly 360 at pivot screw mount 306. As best viewed in the close-up side perspective view of intermediate drawer assembly 360 and top shelf 320 of FIG. 9, chassis release bar 304 has a notch 308 at a back end of chassis release bar 304, which notch 308 receives refrigerator cabinet mounting nut 104. While chassis release bar 304 is pivotable about pivot screw mount 306, a spring 310 biases chassis release bar 304 into a position that engages notch 308 with mounting nut 104, in turn locking chassis 300 in refrigerator cabinet 102. A handle 312 is provided at the front end of chassis release bar 304, which handle 312 may be manually engaged and pushed downward to pivot chassis release bar 304 about pivot screw mount 306, disengaging notch 308 from mounting nut 104, and thus allowing the entire chassis 300 (along with top shelf 320, bottom shelf 340, and drawer assembly 360) to be horizontally pulled outward from refrigerator cabinet 102. Preferably, a release bar cover plate 314 is removably mounted by, for example, security screws to the front face of intermediate drawer assembly 360, allowing an authorized person to access handle 312 to disengage chassis release bar 304 from mounting nut 104 and remove the entire chassis and its contents from the refrigerator cabinet 102.

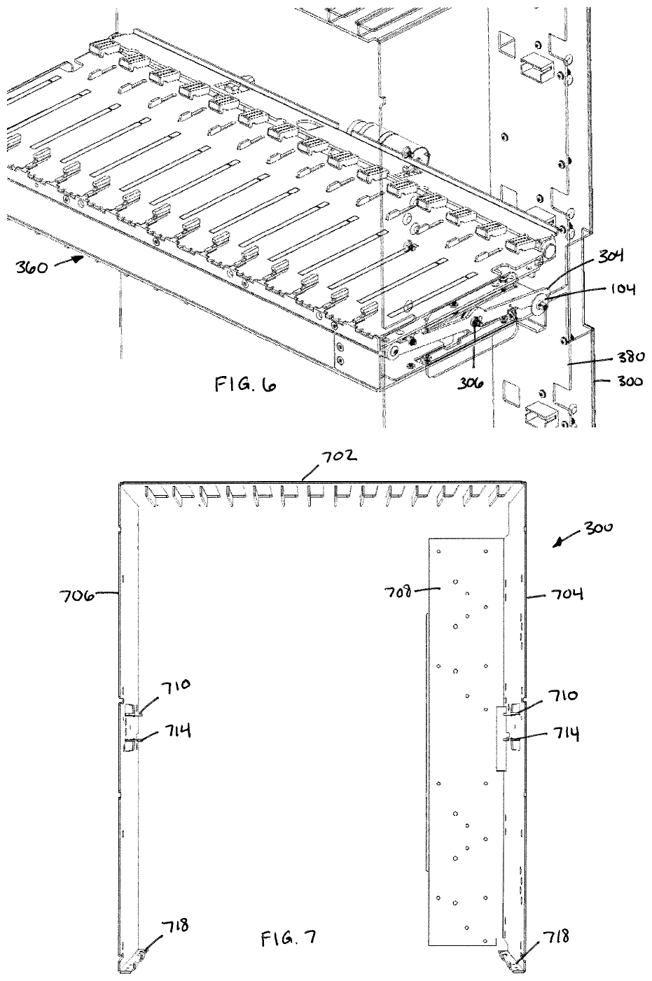

As mentioned above, top shelf 320, bottom shelf 340, and intermediate drawer assembly 360 are mounted to chassis 300. FIG. 7 is a front view of chassis 300 with top shelf 320, bottom shelf 340, and intermediate drawer assembly 360 removed for clarity. Chassis 300 includes a top wall 702, a right side wall 704, and a left side wall 706. Chassis 300 also provides a mounting wall 708 for backplane 380. Right side wall 704 and left side wall 706 include upper support flanges 710 extending into the interior of chassis 300 from each respective sidewall. Upper support flanges 710 receive and support the bottom wall of top shelf 320, such as by way of threaded connectors 712 (FIGS. 8 and 9). Right side wall 704 and left side wall 706 also include lower support flanges 714 extending into the interior of chassis 300 from each respective sidewall. Lower support flanges 714 receive and support the bottom wall of intermediate drawer assembly 360, such as by way of threaded connectors 716. Still further, right side wall 704 and left side wall 706 include bottom mounting brackets 718 extending into the interior of chassis 300 from each respective sidewall. As with upper support flanges 710, bottom mounting brackets 718 receive and support the bottom wall of bottom shelf 342, such as by way of threaded connectors.

Such support configuration allows replacement of any of top shelf 320, bottom shelf 340, or intermediate drawer assembly 360 with minimal effort, as may be desirable to meet changing refrigerated product storage needs. By way of non-limiting example, a given facility may wish to dedicate only bottom shelf 340 to receiving cartridges 400, and may wish to provide alternative storage space in the top portion of chassis 300. In this case, top shelf 320 may be removed from chassis 300 by removing threaded connectors 712, and such alternative storage assembly as may be selected by persons skilled in the art may be affixed in its place. In certain configurations, an open-front storage box equipped with a radio frequency identification (“RFID”) antenna may be put in the place of top shelf 320 and attached to upper support flanges 710, or positioned directly on top shelf 320 or a portion thereof (such as to bottom plate 326 of top shelf 320, discussed below), which may receive and store RFID tagged product containers, as discussed in greater detail below.

With continued reference to FIGS. 7 and 8, top shelf 320 includes a flat panel 321 that serves as the support for product dispensing cartridges 400. In certain configurations, flat panel 321 comprises a printed circuit board. Guide tabs 322 are positioned adjacent a front edge of flat panel 321 and serve to properly position product dispensing cartridges 400 as they are being placed on top shelf 320. To further assist in properly positioning product dispensing cartridges, rear guide posts 325 are likewise provided and positioned in alignment with guide tabs 322, thus defining individual slots 323 configured to receive a product dispensing cartridge 400. Sensors 324 as discussed above are positioned in the top face of flat panel 321 so as to align with a bottom, horizontally moveable dispensing drawer on each cartridge 400, thus allowing sensors on the bottom of such drawers to activate sensors 324 on flat panel 321, in turn allowing the processor of control compartment 115 to record dispensing of product from a particular product dispensing cartridge 400. Top shelf 320 also includes bottom plate 326, which both mounts top shelf 320 to upper support flanges 710, and positions flat panel 321 a vertical distance above bottom plate 326.

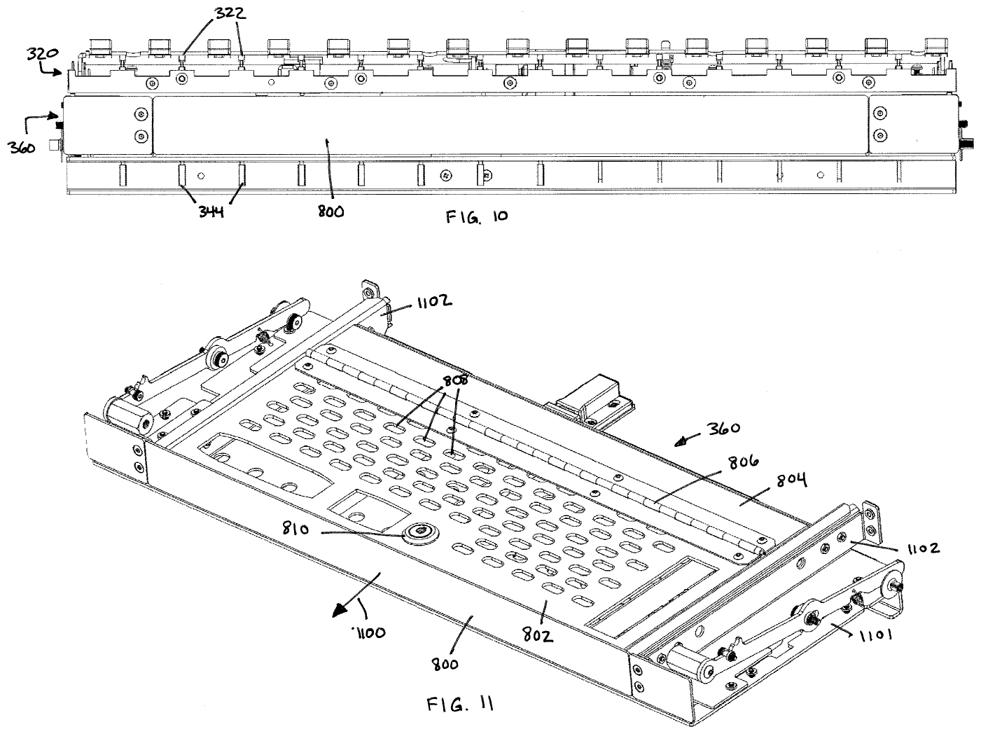

As shown in FIGS. 8-11, while top shelf 320 is positioned immediately above intermediate drawer assembly 360, as discussed above it is independently mounted to chassis 300, allowing drawer assembly 360 to slide horizontally outward (in the direction of arrow 1100 in FIG. 11) from chassis 300 for placement and retrieval of product. As best viewed in FIG. 11 (in which flat panel 321 has been removed for clarity), drawer front 800 may be pulled outward in the direction of arrow 1100 from intermediate drawer assembly 360, such as by way of standard drawer slide rails 1102. Slide rails 1102 are attached to both sides of drawer assembly 360, allowing the drawer to be pulled outward from chassis 300 over drawer assembly support plate 1101 without effecting top shelf 320 or bottom shelf 342, or the dispensing cartridges 400 stored on them. Drawer assembly 360 includes a hinged lid 802, hinged to drawer body 804 via a hinge member 806. Hinged lid 802 is preferably perforated with a plurality of openings 808 allowing cold air to pass through drawer assembly 360 so as to keep contents of drawer assembly 360 cold with the rest of the product stored in refrigerated storage and dispensing unit 100. Hinged lid 802 may be provided a separate lock 810 to provide restricted access to the contents of drawer assembly 360. Drawer assembly 360 may be useful for storing product that was improperly or inadvertently withdrawn from dispensing cartridges, product that has already been dispensed but only partially used, or product in oddly shaped containers that do not fit into dispensing cartridges 400.

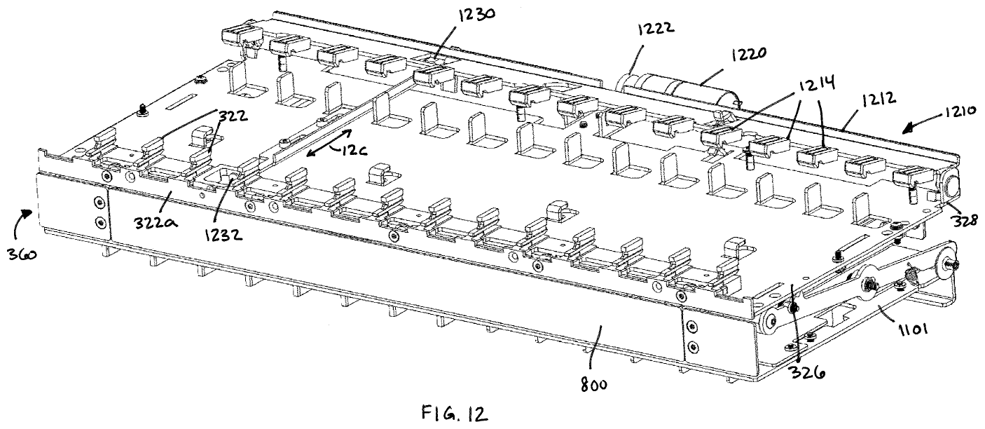

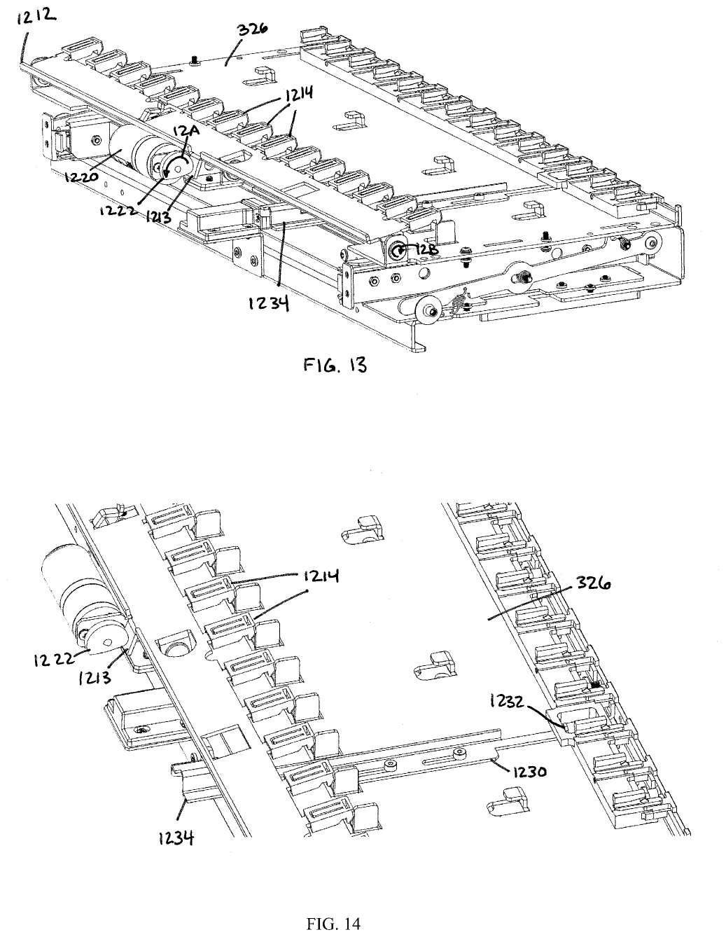

FIG. 12 shows drawer assembly 360 with bottom plate 326 of top shelf 320 in place above drawer assembly 360. Likewise, FIGS. 13 through 16 show drawer assembly with bottom plate 326 removed. Each of FIGS. 12 through 16 show cartridge release mechanism 1210 pivotably mounted to bottom plate 326. More particularly, and with continuing reference to FIGS. 12 through 16, cartridge release mechanism 1210 includes mounting arm 1212 pivotably mounted at pivot tab 328 to bottom plate 326. A plurality of hooks 1214 are mounted to the front face of mounting arm 1212, with one hook 1214 aligned with each slot 323. Hooks 1214 are configured to engage a ridge on the back of each dispensing cartridge 400. Thus, when cartridge release mechanism is in its downward position, hooks 1214 engage the back of dispensing cartridges 400 to prevent their removal from refrigerated storage and dispensing unit 100. When cartridge release mechanism is pivoted to its upward position, hooks 1214 disengage from their respective dispensing cartridges 400, allowing the dispensing cartridges to be removed from their respective shelf 320 and 340. Cartridge release mechanism 1210 is preferably spring biased to its downward position, so as to prevent removal of dispensing cartridges 400 absent an affirmative and authorized action to engage/lift cartridge release mechanism 1210.

In order to cause cartridge release mechanism 1210 to lift hooks 1214, an actuator controlled by the processor, and more particularly a drive motor 1220, is positioned at the back of bottom plate 326. Drive motor 1220 drives a cam wheel 1222, which cam wheel 1222 engages actuator 1213 on mounting arm 1212. Specifically, as cam wheel 1222 is rotated by drive motor 1220 in the direction of arrow 12A (FIG. 13), mounting arm 1212 is rotated in the direction of arrow 12B (FIG. 13) to lower hooks 1214 into their downward, locking position. Likewise, as cam wheel 1222 is rotated in the direction opposite of arrow 12A, mounting arm 1212 is rotated in the opposite direction of arrow 12B to raise hooks 1214 into their upward, unlocked position. Drive motor 1220 is preferably operated by control compartment 115 in response to receiving an instruction from an authorized user to either lock or unlock dispensing cartridges 400 from their respective shelves.

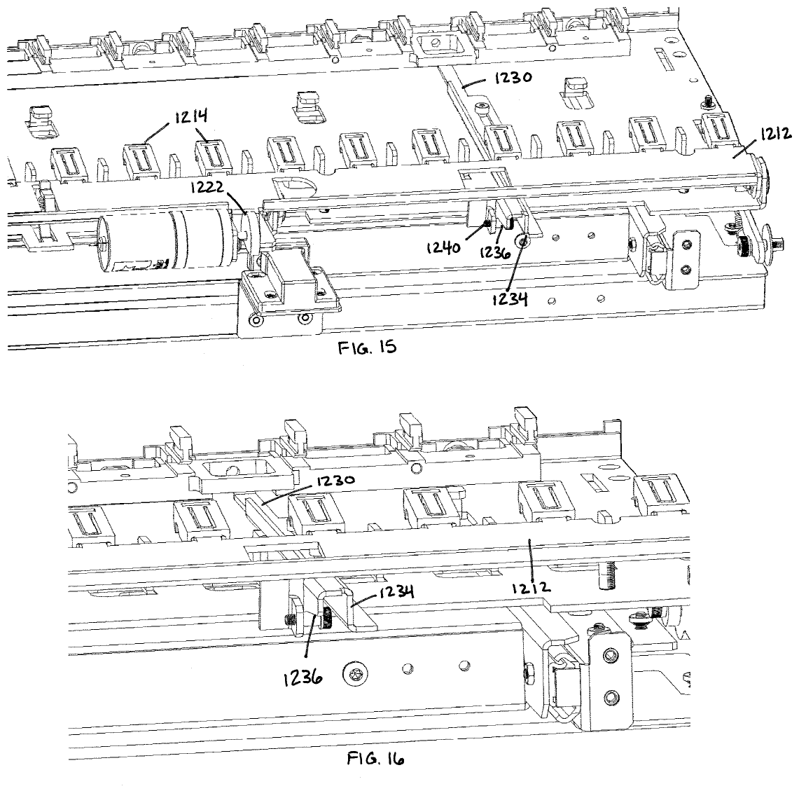

In the event of a power loss, and in the case that during such power loss it is necessary or desirable to remove cartridges 400, a manual release bar 1230 is also provided. Manual release bar 1230 is slidably mounted to the top of bottom plate 326, and is mounted for movement in the direction of arrow 12C (FIG. 12). Release bar 1230 has a pull tab 1232 positioned at the front end of release bar 1230. Preferably, pull tab 1232 is hidden behind the front wall 322a of cartridge guide tabs 322, such that front wall 322a serves as a security panel to prevent unauthorized access to pull tab 1232. Moreover, front wall 322a is preferably mounted to bottom plate 326 with a plurality of security screws, such that one may only access pull tab 1232 with the appropriate tool, and with knowledge that the hidden pull tab 1232 is positioned behind front wall 322a of cartridge guide tabs 322. The back end of release bar 1230 includes a lifting head 1234 that engages the underside of mounting arm 1212. When cam wheel 1222 is rotated so as to allow position hooks 1214 to be pulled to their downward, locking position, release bar 1230 may be pushed to its rearmost position (i.e., the position shown in FIGS. 12 through 16), causing lifting head 1234 to push against mounting arm 1212 and lift mounting arm 1212 and hooks 1214 to their upward, unlocked position, in turn allowing removal of dispensing cartridges 400. As best seen in FIGS. 15 and 16, lifting head 1234 includes a bottom angled edge 1236 that engages a set screw 1240, such that as lifting head 1234 is pushed rearward, bottom angled edge 1236 contacting set screw 1240 causes lifting head 1234 to slightly lift mounting arm 1212, pivoting mounting arm 1212 upward and causing hooks 1214 to raise to their upward, unlocked position.

While FIGS. 12 through 16 show mounting arm 1212 and hooks 1214 and related mechanisms on top shelf 320, an identical assembly is likewise preferably provided for bottom shelf 340 (without intermediate drawer assembly 360).

FIG. 17 shows an exemplary dispensing cartridge 400 for use with the refrigerated storage and dispensing unit 100. Each dispensing cartridge 400 comprises a generally vertical housing 402 and a dispensing drawer 420 at the bottom of housing 402. Dispensing drawer 420 is configured for horizontal sliding out from and back into housing 402. Moreover, each dispensing cartridge 400 includes a dispensing mechanism which allows only a single product unit stored in dispensing cartridge 400 (e.g., a single vial of pharmaceutical product, a single box containing a syringe, etc.) to be dispensed with each pull of dispensing drawer 420, blocks such product unit from being reinserted into dispensing cartridge 400 after dispensing drawer 420 has been pulled out of housing 402, prevents closure of the dispensing drawer until removal of the single product unit from the dispensing drawer 420, and prevents reloading of the next such product unit into the dispensing drawer 420 until dispensing drawer 420 is pushed fully back into housing 402. Each dispensing cartridge 400 is configured to deliver the next product unit by gravity feed into the dispensing drawer 420 after the dispensing drawer 420 has been fully closed following a dispensing operation (i.e., full opening of the dispensing drawer 420 from cartridge 400 and removing the dispensed product unit).

Each dispensing cartridge 400 includes a front face 404, two side walls 406, and a top wall 408. When positioned on one of top shelf 320 and bottom shelf 340, front face 410 of each dispensing cartridge 400 faces the front of refrigerator cabinet 102. A product reload door 410 is accessible from front face 404, which may be opened by a user to reload additional product. However, product reload door 410 has both a vertical portion 410a and a horizontal portion 410b, wherein access to horizontal portion 410b is blocked when dispensing cartridge 400 is fully inserted into refrigerated storage and dispensing unit 100. Thus, a user may only open dispensing cartridge 400, and thus access product stored therein, after the authorized removal of the cartridge 400 from refrigerated storage and dispensing unit 100.

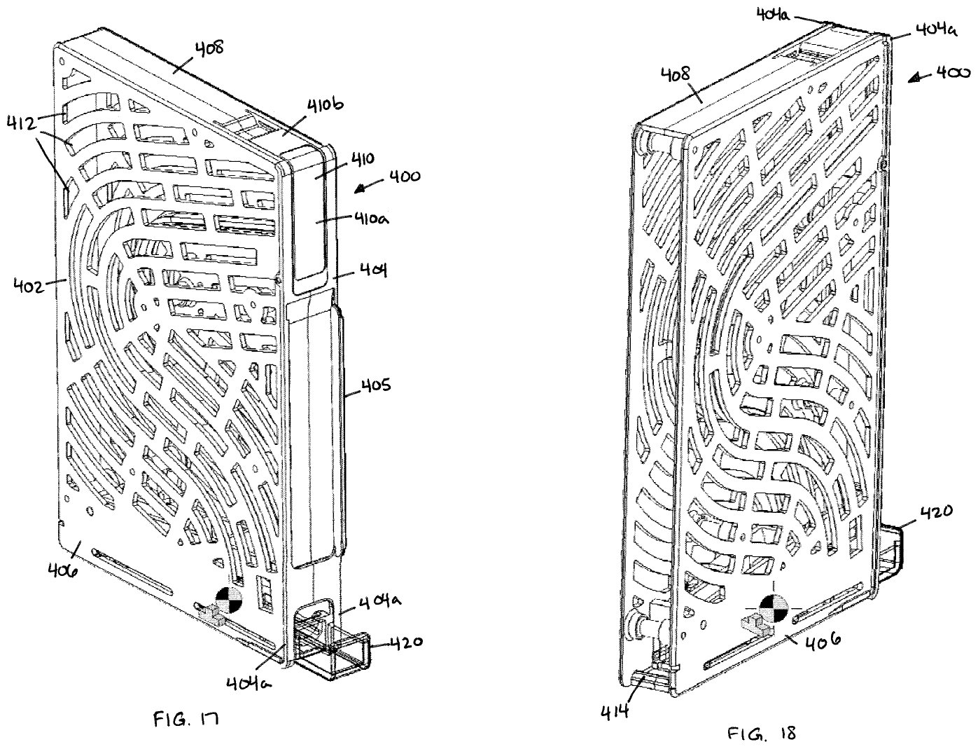

Side walls 406 of dispensing cartridge 400 include a plurality of openings 412. Openings 412, along with the open back (best seen in FIG. 18) of each dispensing cartridge 400, provide airflow around product stored within dispensing cartridge 400 to ensure that all such product remains uniformly refrigerated. Further, front face 404 has a wider width than top wall 408, thus creating a space between adjacent dispensing cartridges 400 when they are positioned on top shelf 320 and bottom shelf 340, further providing for refrigerated airflow in the spaces between adjacent dispensing cartridges 400. The outer edges 404a of front face 404 of adjacent dispensing cartridges 400 abut one another so as to form a relatively uniform front wall with minimal air gap between them, thus keeping as much of the refrigerated air behind the front faces 404 of the dispensing cartridges 400 as possible when the refrigerator cabinet 102 is open. A pull tab 405 may be provided extending vertically along the front face 404 of each dispensing cartridge 400 so as to allow for easy removal of a single dispensing cartridge 400 from top shelf 320 or bottom shelf 340 when desired (and unlocked), such as for refilling purposes.

As best viewed in FIG. 18, horizontal ridge 414 extends across the bottom, rear side of dispensing cartridge 400. Ridge 414 provides an engagement surface against which hooks 1214 of cartridge release mechanism 1210 acts to lock each dispensing cartridge 400 in place on its respective top shelf 320 or bottom shelf 340. More particularly, when hooks 1214 are pivoted to their downward, locking positions, the forward end of a hook 1214 will engage the ridge 414 of an aligned dispensing cartridge 400, thus locking the dispensing cartridge 400 on its respective shelf. Likewise, when hooks 1214 are pivoted to their upward, unlocked position, the forward end of such hook 1214 disengages from the ridge 414 of the aligned dispensing cartridge 400, thus unlocking the dispensing cartridge 400 from its respective shelf and allowing its individual removal from refrigerated storage and dispensing unit 100.

In a particular configuration, dispensing cartridges 400 may include a product sensor that detects the presence of product within dispensing cartridge 400. More particularly, when the dispensing drawer 420 is closed and there is product in the dispensing cartridge 400, the next product to be dispensed may push the front end of a sensing lever downward so as to cause the rear end of such sensing lever to rise, in turn breaking a connection between the product sensor and the sensor on the respective shelf 320 or 340 on which the dispensing cartridge 400 is positioned, thus indicating that there is product in such dispensing cartridge 400 in position for dispensing. When the dispensing drawer 420 is open and there is no additional product pressing the front end of such sensing lever, the rear end of the sensing lever is lowered and connects the product sensor with the sensor on the respective dispensing shelf 320 or 340, indicating that there is currently no product in line to be dispensed. When the dispensing drawer 420 is closed, the next product in line in the dispensing cartridge 400 moves into dispensing drawer 420, pushing down the front end of the sensing lever and causing the rear end of the product sensor to rise and lose its connection with the sensor on the respective dispensing shelf 320 or 340. It is contemplated that other product sensor configurations could be readily implemented by a person of ordinary skill in the art.

In a particular configuration, dispensing cartridges 400 may also include a drawer position sensor located on the bottom of the dispensing drawer 420. The drawer position sensor may connect with a closed position sensor on the respective top shelf 320 and bottom shelf 340 when the dispensing drawer 420 is closed, indicating to the control compartment 115 that the dispensing drawer 420 is closed. The drawer position sensor may also connect with an open position sensor on the respective top shelf 320 and bottom shelf 340 when the dispensing drawer 420 is fully open, indicating to the control compartment 115 that the dispensing drawer 420 is fully open.

Those skilled in the art will recognize that a wide variety of position sensors and product detection sensors may be used with dispensing cartridges 400 and top shelf 320 and bottom shelf 340 of the refrigerated storage and dispensing unit 100 described herein without departing from the spirit and scope of the instant invention.

As mentioned above (and with reference to FIG. 3B), given the modular construction of refrigerated storage and dispensing unit 100 discussed above, instead of dispensing cartridges 400, an alternative storage assembly may be provided as may be selected by persons skilled in the art. As shown in the side, cross-sectional view of FIG. 19, such alternative storage assembly may, in certain configurations, comprise an open-front storage box 500 equipped with a planar radio frequency identification (“RFID”) antenna 550, which may receive and store RFID tagged product containers.

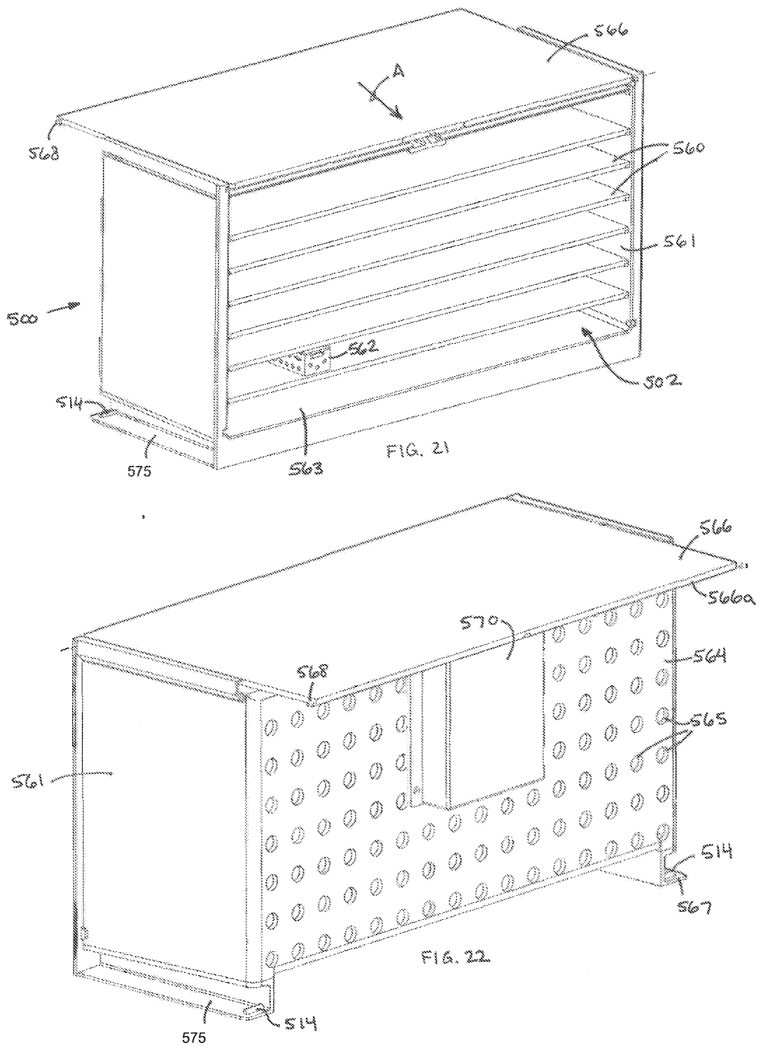

Open-front storage box 500 defines an open interior space 502 that may receive products of varying shapes and sizes, and thus may be used to store products that are not configured for storage in and dispensing from dispensing cartridges 400, but that are nonetheless desired to be stored with such products in a single refrigerated storage and dispensing system 100. With continuing reference to FIG. 19 and to the rear view of FIG. 20, open-front storage box 500 may have a bottom face 504, which bottom face 504 may be sized and otherwise configured to sit directly on flat panel 321 in the place of dispensing cartridges 400, or alternatively on bottom plate 326. Open-front storage box 500 may include ridges 514 on a back side thereof configured to engage with hooks 1214 of cartridge release mechanism 1210, thus allowing open-front storage box 500 to be locked to top shelf 320 or bottom shelf 340 in the same manner that dispensing cartridges 400 may be locked to top shelf 320 or bottom shelf 340. Open-front storage box 500 may include a slot 504 extending into the upper body of open-front storage box 500 from a back side thereof, which slot 504 receives planar RFID antenna 550. An antenna cable 552 may extend from planar antenna 550 out of slot 504 for connection to an RFID reader 570 (FIG. 22), discussed in greater detail below. Planar antenna 550 may be configured to read RFID tags positioned on products or product containers that are to be stored on open-front storage box 500, and more particularly to detect their placement on and removal from open-front storage box 500. Planar RFID antennas and RFID tags readable by them are readily commercially available and their construction is known to those skilled in the art, such that their specific configuration and operation is not further detailed here.

FIGS. 21-23 show an alternative configuration of open front storage box 500. As in FIGS. 19 and 20 discussed above, open front storage box 500 is a modular unit insertable into the refrigerator cabinet as a unit, and includes (as discussed in greater detail below) RFID antennas and an RFID reader embedded in the box structure, thus allowing the entire RFID shelving unit to be placed and removed from the refrigerator cabinet as a single modular unit. Open front storage box 500 may include a plurality of horizontal shelves 560 in open space 502, which shelves extend between wall panels 561. Wall panels 561 may, for example, be grooved to slidably receive the ends of each shelf 560, or may have support ridges 561a to support the ends of shelves 560. The shelves 560 are vertically spaced apart a sufficient distance so as to be able to position a product 562 on the shelf, and more preferably a sufficient distance so as to provide sufficient vertical separation between RFID tags on vertically adjacent product packages 562 to avoid risk of RFID signals interfering with one another. Each product 562 preferably includes an RFID tag allowing the product to be identified by an RFID reader, as discussed below.

Open front storage box 500 also includes a floor panel 563 and a back wall panel 564. Back wall panel 564 preferably includes multiple perforations 565 allowing refrigerated air to flow into the open interior space 502 from the refrigerator cabinet, thus keeping the products 562 stored on shelves 560 in a refrigerated state. A door panel 566 may be provided and positioned to cover the front of storage box 500. Door panel 566 is slidable in the direction of arrow “A” along the top of storage box 500 until the back edge 566a aligns with detents 567 in side wall panels 561. Pin hinge members 568 are affixed to the side ends of back edge 566a of door panel 566, such that when the pin hinge members 568 come to rest in detents 567, door panel 566 may then be pivoted downward to cover the front of storage box 500, in turn thermally insulating the interior of storage box 500. Detents 567 are located within a slot 569 at the top of each wall panel 561, which slot guides the side edges of door panel 566 as it moves across the top surface of storage box 500. A central guide rail 569a may also be provided on the top panel of storage box 500, which may mate with a guide channel (not shown) on the underside of door panel 566 to assist in guiding door panel 566 as it slides across the top panel of storage box 550.

Open front storage box 500 also includes side rails 575 extending horizontally from the front of the storage box 500 to its back, along the bottom of each wall panel 561. The back of each such side rail 575 includes a ridge 514 as described above, allowing hooks 1214 to engage storage box 500 and prevent its unauthorized removal from the refrigerator cabinet. Each side rail 575 is configured to fit within a slot on shelf 320 or 340 defined by guide tabs 322 on each such shelf. An open vertical space is defined between the two side rails 575 and below the bottom, exterior face of floor panel 563, such that the storage box 500 may slide onto either of shelves 320 or 340 directly over the intermediate guide tabs 322.

Storage box 500 is equipped with one or more RFID antennas 572 capable of reading RFID tag information on product package 562, and transferring such data to an RFID reader 570 positioned on the exterior of back wall panel 564 of storage box 500. RFID reader 570 may employ, by way of non-limiting example, an RFID reader module such as an RS2000 RFID Reader Module commercially available from Impinj, Inc., although others may be readily selected and configured for the system described herein by those skilled in the art. One or more cables (not shown) may be provided to interconnect RFID reader 570 with control compartment 115, in turn allowing control compartment to track removal of product packages 562 from storage box 500 and thereby monitor inventory of product within storage box 500. The RFID antennas 572 preferably comprise planar RFID antennas. In a particularly preferred embodiment, one planar antenna 572 is positioned vertically within side wall panel 561 of storage box 500, and one planar antenna 572 is positioned horizontally within floor panel 563 of storage box 500. Each of side wall panel 561 and floor panel 563 may include removable panels that allow access to such planar antennas 572 for servicing. Connecting cables (not shown) may extend from each such antenna 572 to RFID reader 570 on the back of back wall panel 564. It is believed that sufficient tracking capability may be achieved with only two such planar antennas 572 through their placement directly in the structure of storage box 500, thus avoiding the need for a larger number of antennas positioned outside of the refrigerator cabinet 102.

With the foregoing configuration of storage box 500, the entire RFID shelving assembly, including shelves, products, RFID antennas, and RFID reader may be placed into and removed from refrigerator cabinet 102 all as a single, modular unit, thus enabling shipment and placement of pre-stocked storage boxes 500, loading of product packages 562 into storage box 500 with the storage box 500 outside of the refrigerator cabinet 102 (thus avoiding loss of cold air for remaining products in the refrigerator cabinet 102), and removal of storage box 500 and all products 562 stored thereon when necessary, such as in the event of a long-term power loss that results in loss of refrigeration.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. A refrigerated storage and dispensing unit, comprising:
a refrigerator cabinet;
a chassis positioned on an interior of said refrigerator cabinet having walls defining an open cavity on an interior of said chassis;
at least one shelf affixed to said walls of said chassis within said open cavity; and
an open-front storage box removably positioned on said at least one shelf and having a back wall panel, two side wall panels, a floor wall panel, and a top wall panel and defining an open interior space accessible through a front of said storage box, said storage box having a first planar RFID antenna positioned within one of said side wall panels, and a second planar RFID antenna positioned within said floor wall panel, wherein said one of said side wall panels further comprises a first removable section over said first planar RFID antenna, and said floor panel further comprises a second removable section over said second planar RFID antenna, and wherein said RFID antenna and said RFID reader are configured to read RFID tags on products positioned within said open interior space of said open-front storage box;
wherein said open-front storage box further comprises a modular unit configured for insertion into and removal from said chassis.

2. The refrigerated storage and dispensing unit of claim 1, further comprising:
a second shelf affixed to said walls of said chassis within said open cavity; and
a plurality of product dispensing cartridges removably positioned on said second shelf, each said product dispensing cartridge further comprising a vertical housing holding a plurality of product units and a dispensing drawer configured for horizontal movement out of said housing, wherein each said product dispensing cartridge is configured to sequentially dispense a single one of said product units upon each horizontal extension of said dispensing drawer from a fully closed position to a fully open position.

3. The refrigerated storage and dispensing unit of claim 2, each of said first and second shelves further comprising cartridge guide tabs extending upward from each said shelf and defining a plurality of slots, wherein each said slot is sized to receive one of said dispensing cartridges.

4. The refrigerated storage and dispensing unit of claim 3, wherein an exterior face of said floor wall panel is positioned above at least some of said guide tabs on said first shelf

5. The refrigerated storage and dispensing unit of claim 4, wherein each of said first shelf and said second shelf further comprises a plurality of movable latches mounted to a back side of said shelf and configured to engage a ridge on a back said of one of said product dispensing cartridges; said storage box further comprising side rails extending horizontally from a front face of said storage box to a back face of said storage box along a bottom of each said side wall panel, and each said side rail having a ridge on said side rail configured to engage one of said latches.

6. The refrigerated storage and dispensing unit of claim 5, wherein each said side rail is sized to slide within one of said slots on said first shelf and said second shelf.

7. The refrigerated storage and dispensing unit of claim 3, wherein said storage box is slidable onto said first shelf over least some of said slots.

8. The refrigerated storage and dispensing unit of claim 2, further comprising a control compartment and each of said first and second shelves further comprising sensors configured to detect dispensing of product units from one of said dispensing cartridges, wherein said control compartment is in data communication with said sensors and said RFID reader to monitor product inventory within said storage box and said dispensing cartridges.

9. The refrigerated storage and dispensing unit of claim 8, wherein said sensors are configured to detect dispensing of product units from one of said dispensing cartridges upon opening of said dispensing drawer on said one of said dispensing cartridges to a fully open position.

10. The refrigerated storage and dispensing unit of claim 8, said first and second shelves further comprising moveable latches configured to engage said dispensing cartridges and said storage box so as to prevent unauthorized removal of said dispensing cartridges and said storage box from said refrigerated storage and dispensing unit, and wherein said control compartment is in control communication with said latches.

11. The refrigerated storage and dispensing unit of claim 1, further comprising a plurality of removable shelves extending between said side wall panels.

12. The refrigerated storage and dispensing unit of claim 1, wherein said RFID reader is mounted to an exterior of said back wall panel of said storage box.

13. The refrigerated storage and dispensing unit of claim 1, further comprising a door panel slidably mounted to an exterior of said top wall panel of said storage box, and pivotable over a front of said storage box.

14. The refrigerated storage and dispensing unit of claim 1, wherein said chassis is removable from said refrigerator cabinet.

15. The refrigerated storage and dispensing unit of claim 14, further comprising a chassis release bar pivotably mounted on the interior of said chassis and engaging a mounting nut affixed to said interior of said refrigerator cabinet.

16. The refrigerated storage and dispensing unit of claim 15, wherein said chassis release bar is spring biased toward engagement with said mounting nut.

17. The refrigerated storage and dispensing unit of claim 1, wherein said back wall panel includes a plurality of perforations configured to allow airflow from an interior of said refrigerator cabinet into said storage box.
